(19) United States
(12) Patent Application Publication
(10) Pub. No.: US 2011/0136194 A1
(43) Pub. Date: Jun. 9, 2011

(54) METHOD FOR INCREASING ETHANOL YIELD FROM GRAIN

(75) Inventor: Oleg Kozyuk, North Ridgeville, OH (US)

(73) Assignee: ARISDYNE SYSTEMS, INC., Cleveland, OH (US)

(21) Appl. No.: 12/961,597

(22) Filed: Dec. 7, 2010

Related U.S. Application Data
(60) Provisional application No. 61/267,900, filed on Dec. 9, 2009.

(51) Int. Cl. C12P 7/06

(52) U.S. Cl. 435/161

(57) ABSTRACT

A process for increasing ethanol yield from grain comprising mixing grain, water and enzyme to for a grain-based liquid medium. The grain-based liquid medium is passed through a cavitation device at a velocity and pressure capable of generating a cavitation activation energy of at least 0.4 kJ per kilogram of grain-based liquid medium to enhance the activity of the enzyme and increase ethanol yield.

This application claims the benefit of U.S. provisional application Ser. No. 61/267,900 filed Dec. 9, 2009, the contents of which are incorporated herein in their entirety by reference.

### FIELD OF THE INVENTION

The invention relates to a process for producing ethanol, and more particularly, a process for increasing ethanol yield using controlled cavitation and enhanced enzyme activity.

### BACKGROUND OF THE INVENTION

Alcohols are a renewable and clean fuel source. A grain alcohol commonly used as a fuel source is ethanol, which can be produced, in large part, from corn by the fermentation of starch. Generally, ethanol production is accomplished through a fermentation and distillation process wherein starches are released and converted to sugars, then the sugars are converted to alcohol by the addition of yeast. At an industrial level, yeast fermentation processes only convert about one-third of the corn into ethanol.

Ethanol production facilities often begin the production process with a dry or wet milling process. In dry milling, corn, or another suitable grain, is ground up by a hammer or roller mill into a manageable mixture of coarse particles. The dry mixture of particles is combined with water and enzymes to break up the starch from the corn into smaller fragments and then subject the fragments to a saccharification phase wherein the starch is converted to sugar. After the saccharification phase, the resulting sugars are fermented with yeast to facilitate their conversion to ethanol.

Ethanol yield is dependent upon the initial starch content of the corn as well as the availability of the starch to the enzymes that are used in the saccharification process. In conventional processes, the availability of starch is governed, in part, by the success of the milling or similar step in which the corn is broken up into smaller particles. The production processes currently used in commercial ethanol plants are not able to achieve maximum theoretical ethanol yield, thus more corn than theoretically needed must be used to produce a certain amount of ethanol.

In an attempt to increase ethanol yield, the use of cavitation has been included, however it has been limited to reducing the particle size of the feed material for the purposes of, for instance, enhancing subsequent treatment and providing more surface area for enzymatic breakdown of the starches to take place. Additionally, to achieve good particle size reduction, the cavitational forces apply aggressive shear stresses to the grain particles. If the cavitational forces apply too aggressive a shear force in terms of intensity, energy and/or duration, it is possible to cause damage to the components being treated. For example, a significant decrease in the particle size could have an adverse affect on downstream processing steps.

Also, aggressive cavitational forces can degrade desirable proteins and inactivate the enzymes. The collapse of hydrodynamic cavitation bubbles formed by under specific conditions can generate extremely high local pressures and temperatures, which can promote enzyme denaturation. Cavitation can also promote chemical reactions involving H. and OH. free radicals formed by the decomposition of water inside the collapsing bubbles. These free radicals could be scavenged by some amino acid residues of the enzymes participating in structure stability, substrate binding, or catalytic functions.

Accordingly, there is still a need for a process that can obtain a closer to theoretical maximum yield. The method preferably uses a controlled cavitation device to increase enzyme activity and subsequently increase ethanol yield. Ultimately, an enhanced enzymatic bio-conversion process of starches to ethanol could increase domestically produced bio-fuels and decrease importation of foreign oil.

### SUMMARY OF THE INVENTION

The present invention is a process for producing alcohol, more specifically ethanol, from grain wherein the use of cavitation energy to enhance enzyme activity substantially increases the ethanol yield, comprising mixing a grain-based material with water and enzyme to form mashed pre-gelatinized grain-based liquid medium; and subjecting the said grain-based liquid medium to cavitation activation energy not less than 0.44 kJ and not more than 1.56 kJ per kilogram of said grain-based liquid medium at a temperature in the range of 130 F to 190 F.

### BRIEF DESCRIPTION OF THE DRAWINGS

FIG. 1 is a flow diagram of an ethanol production process using cavitation.

FIG. 2 is a cross section view of a controlled flow cavitation apparatus.

FIG. 3 is a cross section view of a controlled flow cavitation apparatus.

### DETAILED DESCRIPTION OF THE INVENTION

Herein, when a range such as 5-25 (or 5 to 25) is given, this means preferably at least 5 and, separately and independently, preferably not more than 25. In an example, such a range defines independently not less than 5, and separately and independently, not more than 25.

The controlled use of cavitational energy to enhance enzyme activity in an ethanol production process can substantially increases the yield of ethanol from corn. Although the exact mechanism by which such cavitational energy enhances enzyme activity, and thus increasing ethanol yield, is not known, there are several possible explanations. For example, the forces obtained from cavitation are used to disaggregate, disassociate, shake off and/or strip away starch granules from protein, and fiber, as well as disassociate tightly packed granules and tightly packed amyloplasts containing starch granules to make them more accessible to an enzyme for subsequent enzymatic treatment. This increase in accessibility may increase enzyme action. Cavitation energy may also enhance the transport of enzyme macromolecules toward the surface of the grain substrate. In another example, absorption of cavitation energy by a protein may produce a transient conformational shift (modifying the 3-dimensional structure) and alter the protein's functional activity. In yet another example, the collapse of cavitation bubbles, which can enhance the removal of hydrolysis reaction products from the reaction zone, may contribute to an overall increase in the reaction rate.

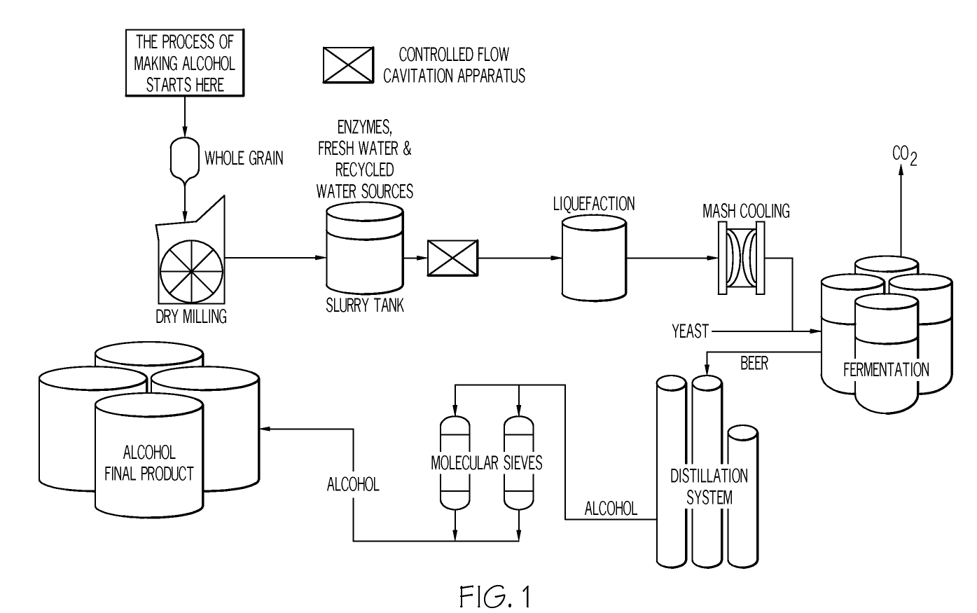

Referring now to the Figures, FIG. 1. shows a starch to ethanol production process, wherein pipes, hoses, or other conventional industrial equipment can be used to facilitate the fluid communication of the elements and streams discussed below. The production process begins when the grain, such as whole kernel corn, is subject to a dry milling phase. The dry milling step is used to grind the grain into meal or powder. Although corn is the whole grain shown in FIG. 1, any suitable grain for producing alcohol can be used. For example, grains can include corn, rye, sorghum, wheat, beans, barley, oats, rice, or combinations thereof. As used herein, the term “grain” can comprise a whole grain or portions or particles of a whole grain such as the product from a dry-milling process used in an alcohol production process.

Next, the grain-based material is mixed with water and enzyme in a slurry mixer to form a pre-gelatinized grain-based liquid medium, which can be in the form of a slurry. The time in which the grain-based material, water, and enzyme are mixed together is preferably in the range of 15 to 60 minutes, for example at least 15, 20, 30, 40, 50 or 60 minutes. The temperature at which the mixing will take place is preferably in the range of 130 to 190° F., for example at least 130, 137, 140, 150, 160, 170, 180, 185 or 190° F. The enzyme added to the pre-gelatinized grain-based liquid medium can be, but is not limited to, alpha-amylase, glucanase, beta-glucosidases, pectinases, xylanase, amylases, lignainases, proteases, beta-mannosidase, and similar enzymes, or a mixture thereof. Enzyme or a mixture of enzymes can be added at a concentration of 0.015 to 0.5 weight percent by weight of grain, such as corn, in the pre-gelatinized grain-based liquid medium, for example enzyme can be added at a concentration of at least 0.015, 0.016, 0.2, 0.28, 0.3, 0.4 or 0.5. For instance, as shown in the Example below, the enzyme can be alpha-amylase and can be present in the grain-based liquid medium in the range of 0.16 to 0.40 weight percent by weight of corn grain in the pre-gelatinized grain-based liquid medium. The grain-based material in the pre-gelatinized grain-based liquid medium can be present at a concentration of 20 to 50 weight percent by weight of the pre-gelatinized grain-based liquid medium, for example, less than 50, 45, 40, 35, 30 or 25 weight percent. Preferably, the grain-based material is present at less than 35 weight percent.

Next, the pre-gelatinized grain-based liquid medium is sent through a cavitation device or apparatus that is used to apply a specified cavitation activation energy to the liquid medium sufficient to activate the enzymes and enhance their activity within the pre-gelatinized grain-based liquid medium. In the processes described herein, enzyme can be added to form the pre-gelantinized grain-based liquid medium without the need for additional enzyme, such as enzyme addition upstream of the process prior to formation of the pre-gelatinized grain-based medium. A one-time addition of enzyme to a grain-based material prior to applying cavitation activation energy, such as through a cavitation device, reduces the need for multiple enzyme additions upstream of liquefaction and increases processing efficiency. For example, enzyme is slurried and mixed with water and grain-based material for less than one hour prior to cavitation. Multiple processing steps prior to cavitation may not be needed, such as long periods of steeping with enzymes, grinding steps, etc. The process therefore can consist of forming mixing a grain-based material, preferably finely ground, with water and enzyme for a period of less than one hour to form a pre-gelatinized grain-based liquid medium prior to application of cavitation activation energy as discussed below.

The cavitation activation energy should be applied at least at a level of about 0.4 kJ per kilogram of grain-based or pre-gelantinized grain-based liquid medium. Preferably, the cavitation activation energy is 0.4 to 1.6 kJ per kilogram of grain-based or pre-gelantinized grain-based liquid medium, for example at least 0.6, 0.8, 1, 1.2 or 1.4 kJ per kilogram. The temperature of the stream of grain-based liquid medium entering the cavitation device can be in the range of 130 to 190° F., for example at least 140, 150, 160, 170 or 180° F. The product exiting the cavitation device can be passed through the cavitation device only one time, or optionally recirculated back through the same cavitation device as many times as desired.

After the pre-gelatinized liquid medium stream passes through the cavitation device it will then move on to the liquidation and cooling phase, as shown in FIG. 1, wherein the enzymes continue to break down the starch polymers of the liquid medium into shorter sections and create a sugar mash. Once the conversion to sugar is complete, the mash will be transferred to fermentation containers or tanks wherein yeast will convert the sugars into carbon dioxide and alcohol, such as ethanol. Upon transfer of the sugar mash to the fermentation containers, additional enzyme, urea, and yeast can be added to the sugar mash. The mixture is then left to ferment for a period of time, for example at least 60 hours. The product resulting from the fermentation process is referred to as “beer” and contains alcohol and solids. These solids can be both soluble and insoluble, such as non-fermentable components left over from the grain. A distillation phase following the fermentation phase separates the liquid carrier, usually water, ethanol, and whole stillage from each other. The water can be recycled and used, for example, in the slurry tanks. The non-fermentable compounds are further separated in the distillation process, and can also be sold as high-protein animal feed.

Adding a cavitation step to the ethanol production process to enhance enzyme activity, wherein parameters such as pressure and temperature can be controlled, can increase ethanol yield. In general, cavitation can be described as the generation, subsequent growth and collapse of cavitation bubbles and cavities. During the collapse of the cavitation bubbles, high-localized pressures and temperatures are achieved, The bubbles contain mostly steam, although the level of steam fluctuates depending on the temperature at which the bubbles are formed. For instance, cavitation bubbles formed at lower temperatures contain less steam. Cavitation bubbles containing less steam collapse more energetically and generate higher local temperatures and pressures. These high temperatures and pressures can stimulate the progress of various chemical reactions which may not be possible under ordinary conditions, such as standard temperature and pressure (STP). However, temperatures and pressures that are too high can have a deleterious effect on a reaction and promote enzyme denaturation. The processing and reaction conditions described below prevent undesirable reactions and minimize enzyme denaturation such that ethanol yield can be increased.

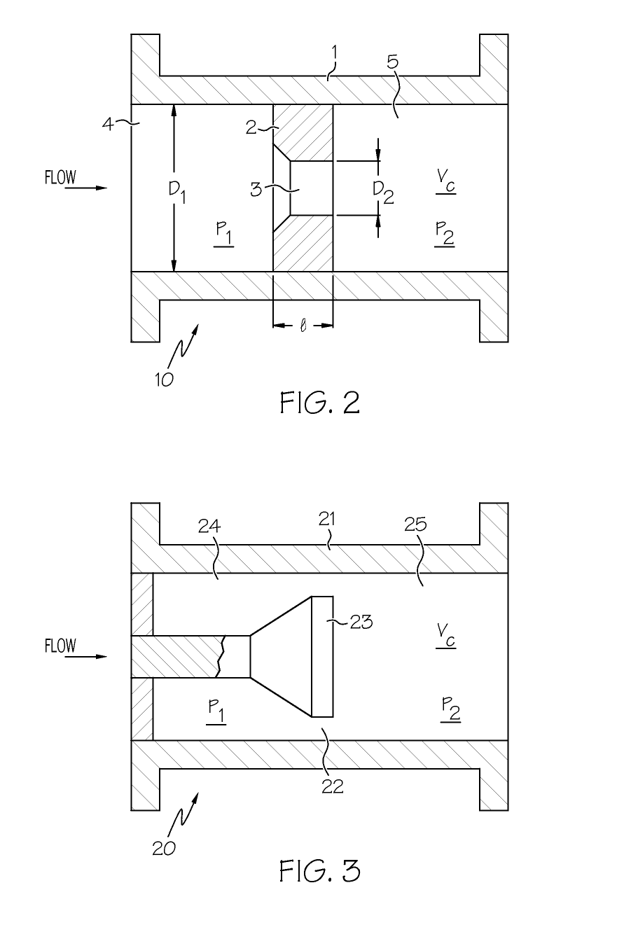

In one embodiment, FIG. 2 illustrates a controlled flow cavitation device. FIG. 2 provides a cross section view of a controlled flow cavitation apparatus 10 which can process a grain-based liquid medium, such as a pre-gelatinized grain-based medium. The controlled flow cavitation apparatus 10 comprises a flow-through channel 1 comprising a first chamber 4 and a second chamber 5. The first chamber 4 and second chamber 5 of the flow-through channel 1 are divided by a localized flow constriction 2. The first chamber 4 is positioned upstream of the localized flow constriction 2 and the second chamber 5 is positioned downstream of the localized flow constriction 2, as viewed in the direction of movement of flow, such as a grain-based liquid medium. Second chamber 5 houses the hydrodynamic cavitation zone as discussed below. The hydrodynamic cavitation zone in the second chamber 5 has volume V_{c}. During operation, the first chamber 4 has static pressure P₁ and the second chamber 5 encompassing the hydrodynamic cavitation zone has static pressure P₂. Localized flow constriction can be achieved by a diaphragm with one, or more, orifices 3.

As shown in FIG. 2, the controlled flow cavitation apparatus 10 comprises one cylindrical orifice 3. The orifice 3 of the apparatus 10 can be any shape, for example, cylindrical, conical, oval, right-angled, square, etc. Depending on the shape of the orifice 3, this determines the shape of the cavitation jets flowing from the localized flow constriction 2. The orifice 3 can have any diameter, D₂, for example, the diameter can be greater thatn 0.1, 1, 2, 3, 5, or 10 mm, and preferably more than 3 mm. In one example, the orifice 3 diameter can be about 3 mm or about 4 mm.

As shown, the first chamber 4 has a pressure P₁ and the second chamber 5 has a pressure P₂. Flow into the apparatus 10 can be provided with the aid of fluid pumping devices as known in the art, such as a pump, centrifugal pump, positive-displacement pump or diaphragm pump. An auxiliary pump can provide flow under a static pressure P₁ to the first chamber 4. As discussed herein, pressure P₁ is defined as the processing pressure for the controlled flow cavitation apparatus 10. The processing pressure is preferably at least 30, 40, 50, 60, 70, 80, 90, 100, 120, 140, 150, 170, 200, 300, 400, 500, 600, 700, 800, 850, 900, or 1000, psi. The processing pressure is reduced as the grain-based liquid medium or pre-gelantinized grain-based liquid medium passes through the flow-through channel 1 and orifice 3. Maintaining a pressure differential across the orifice 3 allows control of the cavitation intensity in the flow through channel 1. The pressure differential across the orifice 3 is preferably at least 30, 40, 50, 60, 70, 80, 90, 100, 120, 140, 150, 170, 200, 300, 400, 500, 600, 700, 800, 850, 900, or 1000, psi. The velocity of the grain-based liquid medium or pre-gelantinized grain-based liquid medium through the orifice 3 in the controlled flow cavitation apparatus is preferably at least 1, 5, 10, 15, 20, 25, 30, 40, 50, 60 or 70 meters per second (m/s).

In the Example below, the controlled flow cavitation apparatus 10 described herein can be used as a single-pass process for enhancing the activity of the enzyme in the pre-gelantinized grain-based liquid medium. Hydrodynamic cavitation arises in the fluid jets flowing from the orifice 3 in the form of intermingling cavitation bubbles and separate cavitation cavities. That is, the orifice 3 creates a hydrodynamic cavitation zone that promotes a high density of cavitation power dissipation locally inside the flow-through channel 1, and more preferably in the orifice 3 chamber and downstream of the orifice 3 in the second chamber 5. The high energy dissipation in the hydrodynamic cavitation zone causes a cavitation activation energy for promoting the activity of the enzymes in the pre-gelatinized grain-based liquid medium for increasing ethanol yield.

The given dynamic pressure and residence time of the bubble or steam bubble in the localized flow constriction 2 allows production of cavitation bubbles and cavities in the liquid flow. The cavity sizes are dependent on the magnitude of the dynamic pressure jet as well as the sizes of orifice 3 in the localized flow constriction 2. Increase of the dynamic pressure jet as well as size of orifice 3 leads to the increase in the sizes of cavitation bubbles. Increase of the dynamic pressure of the cavitation fluid jet also promotes increase of the concentration of cavitation bubbles. Therefore, given the dynamic pressure of the cavitation fluid jet, its shape, and the number of fluid jets, it is possible to produce a cavitation field or zone of cavitation bubbles in the downstream second chamber 5. Cavitation bubbles and cavities together with the liquid jets enter into the second chamber 5, where they collapse under the influence of static pressure P₂. The energy emitted during collapse of cavitation bubbles is directly proportional to the magnitude of the static pressure in the surrounding liquid bubbles. Therefore, the greater the magnitude of P₂ the greater the energy emitted during collapse of cavitation bubbles and the better the dispersion and/or size reduction effect. In other words, the level of energy dissipation in the grain-based fluid medium increases as the magnitude of P₂ increases and thus the severity or hardness of collapse of each cavitation bubble separately increases, as well as the level of energy dissipation due to the decrease of the volume in which these bubbles collapse.

As shown in the Example below, it has been found that cavitation generates a specific cavitation activation energy for promoting the activity of the enzymes. The specified range of cavitation activation energies preferably create hydrodynamic steam cavitation bubbles that collapse less energetically to avoid enzyme denaturation and deleterious effect on a reactions in the alcohol production process. Because cavitation bubbles containing less steam collapse more energetically and generate higher local temperatures and pressures, which can be undesirable, the specified cavitation activation energy, processing temperature and pre-gelatinized grain-based liquid medium make up are believed to create steam-filled hydrodynamic cavitation bubbles that avoid these disadvantages.

The length (l) in orifice 3 in localized flow constriction 2 is selected in such a manner in order that the residence time of the cavitation bubble, for example a hydrodynamic steam cavitation bubble, in the orifice 3 and/or the second chamber 5 is less than 10 seconds, preferably less than 1 second or preferably less than 0.1 second. The time in the hydrodynamic cavitation zone that is needed to enhance and promote the enzyme activity is much smaller than know methods, such as ultrasonic or acoustic, and thus the controlled flow cavitation apparatus can reduce processing time and costs associated with an alcohol production process. Because processing time directly relates to the amount of alcohol that can be produced, the use of a controlled flow cavitation apparatus can increase the yield of alcohol and reduce the amount of processing time required to produce the alcohol. Hydrodynamic cavitation is more efficient than acoustic cavitation and much more efficient than conventional agitation and/or heating methods. Further, the scale-up of hydrodynamic cavitation apparatuses is relatively easy compared to other methods, which makes it well suited to the processing of dispersions and slurries, such as those present in an alcohol production process.

In another embodiment, FIG. 3 provides a cross section view of a cavitation device 20. A bluff body 23 is positioned in the flow-through channel 21 to create a localized flow constrictions 22, wherein two localized flow restrictions are created in parallel to one another, each localized flow restriction positioned between the flow-through channel 21 and the top or bottom of the bluff body 23. The localized flow constrictions, such as the bottom localized flow constriction 22, divide the flow-through channel 21 into two chambers, a first chamber 24 having static pressure P₁ and a second cavitation chamber 25 having static pressure P₂. Second chamber 25 houses the hydrodynamic cavitation zone as discussed below. The hydrodynamic cavitation zone in the second chamber 25 has volume V_{c}.

In operation of the device 20 shown in FIG. 3, liquid, such a the pre-gelatinized grain-based liquid medium, enters the flow-through channel 21 and flow through each localized flow constriction at a pressure and velocity such that a specified cavitation activation energy is generated wherein a hydrodynamic cavitation zone is formed and steam-filled cavitation bubbles are created. The specified range of cavitation activation energies preferably create hydrodynamic steam cavitation bubbles that collapse less energetically to avoid enzyme denaturation and deleterious effect on a reactions in the alcohol production process and thereby enhance the activity in the pre-gelatinized grain-based liquid medium.

The cavitation activation energy through any of the cavitation devices of FIGS. 2-3 can be calculated from the following equation:

$$ɛ = \frac{( P 1 - P 2 ) \cdot Q \cdot t}{ρ \cdot {V}_{C}}$$

wherein ε (kJ/kg) is cavitational energy, P1 (Pa) is the static pressure in the first chamber, P2 (Pa) is the static pressure in the second cavitation chamber, Q (m³/sec) is the flow rate of the liquid medium through the cavitation apparatus, t (sec) is the residence time in the hydrodynamic cavitation zone, Vc (m³) is the volume of the downstream cavitation zone, and ρ (kg/m³) is the density of the pre-gelantinized grain-based liquid medium.

In addition to the pressure differential created by the localized flow restriction 2 in FIG. 2 and bluff body 23 in FIG. 3, the collapse of the cavitation steam bubbles also generates local pressure differentials and lower-energy shock waves. This additional agitation acts to greatly improve the enzymes' effectiveness by significantly increasing their reaction rate without destroying the enzymes. Collapsing hydrodynamic cavitation steam bubbles under elevated static pressure can avoid generating high-temperature zones and the formation H. and OH. free radicals.

Examples of static cavitational energy sources that can be used to apply cavitational energy to the pre-gelatinized grain-based liquid medium include, but are not limited to, static mixers, orifice plates, perforated plates, nozzles, venturis, jet mixers, eductors, cyclonettes (e.g., Fluid-Quip, Inc.), and control flow cavitation devices (e.g., Arisdyne systems, Inc), such as those described in U.S. Pat. Nos. 5,810,052; 5,931,771; 5,937,906; 5,971,601; 6,012,492; 6,502,979; 6,802,639; 6,857,774 and 7,667,082. Additionally, the dynamic cavitational energy sources that can be used include, but are not limited to, rotary milling devices (e.g., EdeniQ Cellunator™), rotary mixers (e.g., HydroDynamics SPR, Magellan™), rotor-rotor (e.g., Eco-Fusion Canada Inc.) and rotor-stator devices (e.g., IKA® Works, Inc., Charles Ross & Son Company, Silverson Machines, Inc., Kinematica Inc.), such as those described in U.S. Pat. Nos. 6,857,774; 7,178,975; 5,183,513; 5,184,576; 5,239,948; 5,385,298; 5,957,122; and 5,188,090.

Achieving increased alcohol yield within a particular type of cavitation process however, is dependent on many factors, including the location of the process at which the cavitation is applied, intensity of the cavitation, duration of time spent in hydrodynamic cavitation zone, pressure maintained in cavitation chamber, temperature, amount of enzyme, and others process variables.

In order to promote a further understanding of the invention, the following Example is provided. This Example is shown by way of illustration and not limitation.

### Example

Corn flour was fed into a slurry mixer where it was mixed with hot process water. Total dry solids concentration was of 30.9% (w/w). Residence times in the slurry mixer were 30 minutes. A dose of α-amylase was included in the mixture that was supplied to the slurry mixer (0.016% w/w enzyme based on the weight of corn flour in the slurry) such that a pre-gelatinized grain-based liquid medium was formed. The temperature, level and pH of the slurry were continuously measured using online instrumentation. Next, the pre-gelatinized grain-based liquid medium was passed from the slurry mixer to a cavitation device as illustrated in FIG. 2. The pre-gelatinized grain-based liquid medium was treated by cavitation at one of two temperatures (137° F. and 170° F.) and one of four cavitation activation energies (0.00, 0.44, 0.94, and 1.56 kJ per kilogram of the pre-gelatinized grain-based liquid medium), as shown in Table 1. The pre-gelatinized grain-based liquid medium was passed through the cavitation device one time as a single-pass operation. The cavitation device had an orifice of 5 mm. Flowrates of the pre-gelatinized grain-based liquid medium ranged from 10 to 18 gpm. Pressure in the first chamber was 100, 200 and 300 psi and static pressure in the second chamber was at least 50 psi. Duration of the pre-gelatinized grain-based liquid medium in the hydrodynamic cavitation zone was less than 0.1 second.

The resulting liquid mixture that was produced after traveling through the cavitation device was discharged to a portable collection tank. Samples of the mixture were collected from this tank in 1-liter bottles and immediately taken to the fermentation laboratory. Once in the fermentation laboratory, an overhead agitator was used to continuously stir the samples to ensure that the corn solids stayed in suspension. While still stirring the samples with the agitator, 160 grams of the mixture was pumped from each of the sample bottles into tarred, sterile, 250-ml Erlenmeyer flasks using a peristaltic pump. Prior to filling, the flasks were weighed to determine their total mass.

Once the mixture was transferred to the flasks, the flasks were left to incubate for 1 hour at 180° F. Subsequently, the flasks were transferred to an incubator shaker to facilitate the cooling of the samples, wherein the temperature was held to 68° F. and the flasks were shaken at 150 rpm. After all of the samples were liquefied and cooled, glucoamylase, urea, and yeast nutrients were added to the flasks. The samples were then left to ferment for at least 60 hours.

After completion of this process, the total mass of each fermentation flask, including beer, was measured and compared to the initial mass of each fermentation flask. The concentration of ethanol was then measured by HPLC. Results are shown in Table 1 below.

TABLE 1

| Tempera- | Cavitation | Enzyme | Ethanol | Increase |
|---|---|---|---|---|
| ture, | activation energy, | concentration, | concentration, | ethanol |
| ° F. | ε kJ/kg | % w/w | g/100 ml | yield, % |
| 170 | none | 0.016 | 13.05 | — |
| 170 | none | 0.028 | 13.09 | +0.30% |
| 170 | none | 0.040 | 13.11 | +0.45% |
| 170 | 0.44 | 0.016 | 13.11 | +0.45% |
| 170 | 0.94 | 0.016 | 13.37 | +2.45% |
| 170 | 1.56 | 0.016 | 13.44 | +2.99% |
| 137 | none | 0.016 | 13.04 | — |
| 137 | none | 0.028 | 12.91 | — |
| 137 | none | 0.040 | 12.87 | — |
| 137 | 0.44 | 0.016 | 12.95 | — |
| 137 | 0.94 | 0.016 | 13.29 | +1.92 |
| 137 | 1.56 | 0.016 | 13.23 | +1.46% |

The experimental data demonstrated that introduction of specified cavitation activation energy from at least 0.44 to 1.56 kJ per kilogram of grain-based or pre-gelantinized grain-based liquid medium into the pre-gelatinized grain-based liquid medium containing enzymes can improve the effectiveness of the enzymes so that the ethanol yield from grains is increased. As can be seen, the lower temperature is less effective with respect to enzyme activation, for example the processing temperature of 137° F. generally yielded a lower increase in ethanol as compared to the results at the processing temperature of 170° F. This result is mostly likely due to the fact that the bubbles were formed at lower temperatures, thus containing less steam in the bubbles which caused them to collapse more energetically and generate higher local pressures and temperatures. This sequence of events can promote the formation of free-radicals, which can have a negative effect on the catalytic function of the enzymes, thus explaining the lower relative yields.

It should now be apparent that there has been provided, in accordance with the present invention, a novel process for enhancing enzyme activity in grain-based liquid medium that satisfies the benefits and advantages set forth above. Moreover, it will be apparent to those skilled in the art that many modifications, variations, substitutions and equivalents for the features described above may be effected without departing from the spirit and scope of the invention. Accordingly, it is expressly intended that all such modifications, variations, substitutions and equivalents which fall within the spirit and scope of the invention as defined in the appended claims to be embraced thereby.

The preferred embodiments have been described, herein. It will be apparent to those skilled in the art that the above methods may incorporate changes and modifications without departing from the general scope of this invention. It is intended to include all such modifications and alterations in so far as they come within the scope of the appended claims or the equivalents thereof.

What is claimed is:

1. A process for increasing ethanol yield from grain comprising the steps of:
(a) mixing a grain-based material with water and enzyme for a period of 15 to 60 minutes at a temperature of 130 to 190° F. to form a pre-gelatinized grain-based liquid medium; and
(b) subjecting said grain-based liquid medium at a temperature of 130 to 190° F. to cavitation activation energy not less than 0.4 kJ per kilogram of said grain-based liquid medium to enhance the activity of said enzyme in said grain-based liquid medium.

2. The process of claim 1, said grain-based material being selected from the group consisting of corn, rye, sorghum, wheat, beans, barley, oats, rice, or combinations thereof.

3. The process of claim 1 said enzyme being selected from the group consisting of alpha-amylase, glucanase, beta-glucosidases, pectinases, xylanase, amylases, lignainases, proteases, [beta]-mannosidase, and mixtures thereof.

4. The process of claim 1, said enzyme being present in said pre-gelatinized grain-based liquid medium at a concentration of more than 0.015 weight percent by weight of grain-based material.

5. The process of claim 1, said grain-based material being present in said pre-gelatinized grain-based liquid medium at a concentration of less than 40 weight percent by weight of said pre-gelatinized grain-based liquid medium.

6. The process of claim 1, said cavitation activation energy being generated by localized hydrodynamic steam cavitation bubbles collapsing under elevated static pressure.

7. The process of claim 1, wherein the cavitation activation energy is produced by static or dynamic cavitation means.

8. The process of claim 7, wherein said static means is at least one device selected from the group consisting of static mixers, orifice plates, perforated plates, nozzles, venturis, jet mixers, eductors, cyclones and control flow cavitation devices.

9. The process of claim 7, wherein said dynamic means is at least one device selected from the group consisting of rotary mixers, rotary milling devices, rotor-rotor and rotor-stator devices.

10. A process for increasing ethanol yield from grain comprising the steps of:
(a) mixing a grain-based material with water and enzyme for a period of 15 to 60 minutes at a temperature of 130 to 190° F. to form a pre-gelatinized grain-based liquid medium; and
(b) said pre-gelatinized grain-based liquid medium being passed through a controlled flow cavitation apparatus at a temperature of 130 to 190° F. and at a processing pressure capable of generating an cavitation activation energy not less than 0.4 kJ per kilogram of said grain-based liquid medium to enhance the activity of said enzyme in said grain-based liquid medium, wherein said cavitation activation energy is generated in a hydrodynamic cavitation zone.

11. The process of claim 10, said pre-gelatinized grain-based liquid medium being maintained in said hydrodynamic cavitation zone for less than 10 seconds.

12. The process of claim 10, said pre-gelatinized grain-based liquid medium being passed through said controlled flow cavitation apparatus at least one time.

13. The process of claim 10, said enzyme being present in said pre-gelatinized grain-based liquid medium at a concentration of more than 0.015 weight percent by weight of grain-based material.

14. The process of claim 10, said grain-based material being present in said pre-gelatinized grain-based liquid medium at a concentration of less than 40 weight percent by weight of said pre-gelatinized grain-based liquid medium.

15. The process of claim 10, said cavitation activation energy being generated by localized hydrodynamic steam cavitation bubbles collapsing under elevated static pressure is said hydrodynamic cavitation zone.
